(19) United States
(12) Patent Application Publication
(10) Pub. No.: US 2007/0003511 A1
(43) Pub. Date: Jan. 4, 2007

(54) Lipid combination to combat ashy skin

(75) Inventors: Jens Schulz, Schenefeld (DE); Stephanie von der Fecht, Wedel (DE); Astrid Heptner, Hamburg (DE); Stefanie Kaninck, Hamburg (DE); Frank Schwanke, Hamburg (DE)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 11/083,549

(22) Filed: Mar. 18, 2005

(30) Foreign Application Priority Data
Mar. 19, 2004 (DE) 10 2004 013 607.6

(51) Int. Cl. A61K 8/97; A61K 36/898

(52) U.S. Cl. 424/74; 424/776

(57) ABSTRACT

The present invention is a water-in-oil emulsion comprising a combination of shea butter and cocoa butter and a method of using same to combat dry, cracked or ashy skin, particularly highly pigmented skin.

### FIELD OF THE INVENTION

The present invention relates to a combination of shea butter and cocoa butter, and to the use thereof.

### BACKGROUND OF THE INVENTION

The desire to look beautiful and attractive is naturally rooted in humans. Although the beauty ideal has undergone changes over the course of time, the achievement of a flawless outer appearance has always been the aim of humans. An essential part of a beautiful and attractive appearance is the condition and the appearance of the skin.

The skin is the largest human organ. Amongst its many functions (for example temperature regulation and as a sensory organ), the barrier function, which prevents the organism from drying out, is probably the most important. At the same time, the skin acts as a protective device against the penetration and absorption of external substances. This barrier function is effected by the epidermis which, being the outermost layer, forms the actual protective sheath against the environment. Being about one tenth of the total thickness, it is also the thinnest layer of the skin.

The skin is subjected to stresses through various exogenous and endogenous factors; these impair its external appearance and its physiological function. One result of these impairments is, for example, a drying out of the skin. The cause of dry skin is usually a lack of moisturizing factors (moisturizers) in the epidermis. In the case of atopically dry skin, a disturbed fatty acid metabolism additionally plays a role. An important factor of dry skin is the reduced water-binding capacity. This depends on the concentration of natural moisturizing factors (NMF) in the skin, with epidermal lipids playing an essential role besides urea and amino acids. A lack of natural moisturizing factors leads to an increased transepidermal water loss (TEWL) and ultimately to the drying out of the skin with its typical symptoms such as roughness, flaking, taut feeling, cracking, reddening or itching. The lack of natural moisturizing factors can be caused by endogenous factors, such as genetic disposition, hormonal imbalances, disorders or as a result of exogenous factors such as degreasing of the skin during skin cleansing or as a result of climatic effects.

Dry skin troubles an increasing number of patients particularly during winter. As well as elderly people and children of pale skin type, people with a darker skin type are also affected overproportionally often by this complaint.

Depending on the origin and descent, various skin types can be observed in humans since this organ has adapted over the course of evolution to the various environmental influences. Thus, the division of the skin into the following six pigmentation types with varying sensitivity to UV light radiation is known, for example, from the field of sun protection:

| Skin | | |
|---|---|---|
| type | Description | Designation |
| I | Skin remarkably light, pale and | Celtic type |
| | predisposed to freckles; reddish | |
| | hair; nipples: very pale; eyes: | |
| | green, blue | |
| II | Somewhat darker than Type I, | Germanic type |
| | occasionally freckled; hair | |
| | blonde to brown; nipples: light; | |
| | eyes: blue, green | |
| III | Skin light to light brown, no | Dark-skinned European |
| | freckles; hair dark blonde, | |
| | brown; nipples: darker | |
| IV | Skin brown, olive; no freckles; | Mediterranean type |
| | hair: dark brown, black; | |
| | nipples: very dark | |
| V | Skin with deep-brown base colour | Middle Eastern or |
| | | South-American type |
| VI | Skin with dark brown to black | African type |
| | colour | |

Although in itself obvious, in cosmetic and dermatological skincare it has for a long time gone largely unnoticed that different skin types also react with varying sensitivity to other environmental influences such as heat, cold, dryness or high atmospheric humidity. Only recently has it been attempted to take this phenomenon into consideration with the help of “ethnic skincare” or “ethnic cosmetics”. In this connection, the problems of people with skin types IV to VI, especially in moderate and cold climatic zones, have also increasingly become the focus of research. As rich is the supply for cosmetics for pale-skinned people, which protect, for example, against the negative effects of exposure to UV light, so the supply for dark-skinned people to protect in particular their skin against the stresses resulting from cold and dry air is hitherto inadequate.

In cases of highly pigmented skin, a drying out of the skin leads to an optical grey coloration thereof. Dead dried-out flakes of skin produce a grey film which is generally perceived as being not very aesthetic. In Anglo-Saxon literature, this phenomenon is known under the term “ashy skin”. According to the prior art, this “ashy skin” is treated with pure fatty creams, e.g., like vaseline. However, such products have the disadvantage that they produce an intensive greasy-sticky feel on the skin, impart to it a shiny unaesthetic appearance and in most cases cover the skin such that it is air-impermeable. The ability to store moisture in the skin is also extremely limited. Such preparations can confidently be referred to as “stone age cosmetics”.

### SUMMARY OF THE INVENTION

It was therefore the object of the present invention to develop lipid combinations and preparations which overcome the shortcomings of the prior art and permit effective prophylaxis, treatment and care of dry or really dry cracked skin and in particular highly pigmented dry or really dry cracked skin. The rough, tight feel of the skin which arises in cases of dry skin was likewise to be overcome effectively and permanently, such as the marks, cracks and/or reddening of the skin which arise in cases of dry skin.

Surprisingly, the objects are achieved by
a cosmetic and/or dermatological water-in-oil emulsion comprising a lipid combination of shea butter and cocoa butter.
the use of a lipid combination of shea butter and cocoa butter for the prophylaxis, treatment and care of dry and/or cracked skin, in particular of highly pigmented dry and/or cracked skin.
the use of a W/O emulsion comprising a lipid combination of shea butter and cocoa butter for the prophylaxis, treatment and care of dry and/or cracked skin, in particular of dry and/or cracked highly pigmented skin.
the use of a lipid combination of shea butter and cocoa butter for the prophylaxis, treatment and care of the phenomenon of ashy skin in people with highly pigmented skin.
the use of a W/O emulsion comprising a lipid combination of shea butter and cocoa butter for the prophylaxis, treatment and care of the phenomenon of ashy skin in people with highly pigmented skin.

Here, “highly pigmented skin” or “dark-skinned” are understood according to the invention in particular as meaning skin types of category IV-VI.

### DETAILED DESCRIPTION OF THE PREFERRED EMBODIMENTS

Cocoa butter (cocoa oil, INCI Theobroma Cacao (Cocoa) Seed, CAS 8002-31-1) is the name referred to the fat which is yellowish in colour and has a weak cocoa odour that is formed during cocoa processing and located in the storage seed leaves of the cocoa bean (50-58%). Cocoa butter forms six crystal forms with melting points between 17.3 and 36.3° C. It consists on average of 25% palmitic acid, 37% stearic acid, 34% oleic acid and 3% linoleic acid. The fatty acid profile is characterized by narrow concentration ranges; only for linoleic acid do slight differences arise. Among the vitamin E derivatives, the γ-tocopherol usually predominates, the content of which, however, is subject to very considerable fluctuations. On account of the climatic requirements of the cocoa plant (Theobroma cacao L., Sterculiaceae), cocoa is cultivated exclusively in a narrow belt along the equator. For this reason it is possible to differentiate three main provenances (Central and South America, West Africa, South East Asia/Oceana), which are also revealed in the chemical composition of the cocoa butter. A high content of symmetrical monounsaturated triglycerides is responsible for many of the characteristic properties of cocoa butter (Römpp Chemie-Lexikon online, Version 2.2).

Shea butter (INCI Butyrosspermum Parkii (shea butter), CAS 68920-03-6) is obtained from the seeds (about 45% fat) of the approximately 20 m-tall shea Vitellaria paradoxa, [syn. Butyrospermum parkii], Sapotaceae, native primarily to West Africa. It is a viscous-butter-like fat which is relatively stable to oxidation and is used directly for nutritional purposes in the countries of origin (e.g. Sudan). In addition, shea butter is used in cosmetics. The most important fatty acids in the triglycerides are: oleic acid 49-50%, stearic acid 36-42%, palmitic acid 5-6% and linoleic acid 4-5%. The export amounts are about 50 000 t/a (Römpp Chemie-Lexikon online, Version 2.2).

Uses and water-in-oil emulsions (W/O emulsions) advantageous according to the invention are characterized in that they comprise cocoa butter in an amount of from 0.1 to 30% by weight, and preferably in an amount of from 0.5 to 10% by weight, in each case based on the total weight of the preparation.

Uses and water-in-oil emulsions (W/O emulsions) advantageous according to the invention are characterized in that they comprise shea butter in an amount of from 0.1 to 30% by weight and preferably in an amount of from 0.5 to 30.0% by weight, in each case based on the total weight of the preparation.

Uses and water-in-oil emulsions advantageous according to the invention are also characterized in that the weight ratio of shea butter to cocoa butter in the emulsion is from 1:1 to 1:10.

In this connection, it is advantageous according to the invention if the W/O emulsifiers used are one or more compounds chosen from the group consisting of polyglycerol isostearates and ethoxylated W/O emulsifiers, with the following emulsifiers/emulsifier mixtures being preferred according to the invention: triglycerol diisostearate and PEG-40 perisostearate and triglycerol diisostearate and diglycerol dipolyhydroxystearate.

Uses and water-in-oil emulsions advantageous according to the invention are characterized in that the W/O emulsifiers are present in the emulsion in a total amount of from 0.1 to 15% by weight, and preferably in a total amount of from 0.75 to 5% by weight, in each case based on the total weight of the preparation.

The lipid phase according to the invention of the emulsion can comprise fats, oils, waxes and other fatty bodies.

The lipid phase can advantageously be chosen from the following group of substances:
mineral oils, mineral waxes
oils, such as triglycerides of capric acid or of caprylic acid, and also natural oils, such as, for example, castor oil, macadamia oil, avocado oil or jojoba oil, dialkyl ethers, such as, for example, di-n-octyl ethers, and dialkyl carbonates, such as, for example, di-n-octyl carbonate
fats, waxes and other natural and synthetic fatty bodies, preferably esters of fatty acids with alcohols of low carbon number, e.g. with isopropanol, propylene glycol or glycerol, or esters of fatty alcohols with alkanoic acids of low carbon number or with fatty acids;
alkyl benzoates;
silicone oils, such as dimethylpolysiloxanes, diethylpolysiloxanes, diphenylpolysiloxanes, and mixed forms thereof.

According to the invention, it can also advantageously comprise compounds chosen from the group of esters of saturated and/or unsaturated, branched and/or unbranched alkanecarboxylic acids of chain length from 3 to 30 carbon atoms and saturated and/or unsaturated, branched and/or unbranched alcohols of chain length from 3 to 30 carbon atoms, from the group of esters of aromatic carboxylic acids and sturated and/or unsaturated, branched and/or unbranched alcohols of chain length from 3 to 30 carbon atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyidodecyl palmitate, oleyl oleate, oleyl erucate, erucyl oleate, erucyl erucate, and synthetic, semisynthetic and natural mixtures of such esters, e.g. jojoba oil.

In addition, the oil phase can advantageously be chosen from the group of branched and unbranched hydrocarbons and hydrocarbon waxes, silicone oils, dialkyl ethers, the group of saturated or unsaturated, branched or unbranched alcohols, and fatty acid triglycerides, namely the triglycerol esters of saturated and/or unsaturated, branched and/or unbranched alkanecarboxylic acids of chain length from 8 to 24, in particular 12-18, carbon atoms. The fatty acid triglycerides can, for example, be chosen advantageously from the group of synthetic, semisynthetic and natural oils, e.g. olive oil, sunflower oil, soya oil, peanut oil, rapeseed oil, almond oil, palm oil, coconut oil, palm kernel oil and the like.

Any mixtures of such oil and wax components can also be used advantageously for the purposes of the present invention. In some cases, it may also be advantageous to use waxes, for example cetyl palmitate, as the sole lipid component of the oil phase.

The oil phase is advantageously chosen from the group consisting of 2-ethylhexyl isostearate, octyldodecanol, isotridecyl isononanoate, isoeicosane, 2-ethylhexyl cocoate, C₁₂₋₁₅-alkyl benzoate, caprylic/capric triglyceride, dicaprylyl ether.

Mixtures of C₁₂₋₁₅-alkyl benzoate and 2-ethylhexyl isostearate, mixtures of C₁₂₋₁₅-alkyl benzoate and isotridecyl isononanoate and mixtures of C₁₂₋₁₅-alkyl benzoate, 2-ethylhexyl isostearate and isotridecyl isononanoate are particularly advantageous.

Of the hydrocarbons, paraffin oil, squalane and squalene are to be used advantageously for the purposes of the present invention.

The oil phase can also advantageously have a content of cyclic or linear silicone oils, or consist entirely of such oils, although it is preferred, apart from the silicone oil or the silicone oils, to use an additional content of other oil phase components.

Cyclomethicone (octamethylcyclotetrasiloxane) is advantageously used as silicone oil to be used according to the invention. Other silicone oils, however, are also to be used advantageously for the purposes of the present invention, for example hexamethylcyclotrisiloxane, polydimethylsiloxane, poly(methylphenylsiloxane).

Also particularly advantageous are mixtures of cyclomethicone and isotridecyl isononanoate, and of cyclomethicone and 2-ethylhexyl isostearate.

The aqueous phase of the preparations according to the invention optionally advantageously comprises alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, and also alcohols of low carbon number, e.g. ethanol, isopropanol, 1,2-propanediol, 2-methyl-1,3-propanediol, glycerol and in particular one or more thickeners which can be chosen advantageously from the group consisting of silicon dioxide, aluminium silicates, polysaccharides and derivatives thereof, e.g. hyaluronic acid, xanthan gum, hydroxypropylmethylcellulose.

The cosmetic and dermatological preparations according to the invention can comprise cosmetic auxiliaries as are customarily used in such preparations, e.g. preservatives, bactericides, perfumes, UV photoprotective filters, skin-bleaching agents, substances for preventing foaming, dyes, pigments which have a colouring effect, thickeners, surface-active substances, emulsifiers, softening, moisturizing and/or humectant substances, fats, oil, waxes or other customary constituents of a cosmetic or dermatological formulation, such as alcohols, polyols, polymers, foam stabilizers, electrolytes, organic solvents or silicone derivatives.

In particular, active ingredient combinations used according to the invention can also be combined with other antioxidants and/or free-radical scavengers.

Such antioxidants are advantageously chosen from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. α-carotene, β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof), and salts thereof, dilauryl thiodipropionate, distearyl thiodipropionate, thiodipropionic acid and derivatives (esters, ethers, peptides, lipids, nucleotides, nucleosides and salts) thereof, and sulfoximine compounds (e.g. buthionine sulfoximines, homocysteine sulfoximine, buthionine sulfones, penta-, hexa-, heptathionine sulfoxamine) in very low tolerated doses (e.g. pmol to μmol/kg), and also (metal) chelating agents (e.g. α-hydroxy fatty acids, palmitic acid, phytic acid, phytin, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, tocopherols and derivatives (e.g. vitamin E acetate), vitamin A and derivatives (vitamin A palmitate), and coniferyl benzoate of benzoin resin, rutinic acid and derivatives thereof, butylhydroxytoluene, butylhydroxyanisol, nordihydroguaiacic acid, nordihydroguaiaretic acid, trihydroxybutyrophenone, uric acid and derivatives thereof, mannose and derivatives thereof, sesamol, sesamolin, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives of these active ingredients mentioned which are suitable according to the invention (salts, esters, ethers, sugars, nucleotides, nucleosides, peptides and lipids).

The amount of the abovementioned antioxidants (one or more compounds) in the preparations is preferably 0.001 to 30% by weight, particularly preferably 0.05-20% by weight, in particular 1-10% by weight, based on the total weight of the preparation.

If vitamin E and/or derivatives thereof are the additional antioxidant or antioxidants, it is advantageous to choose their particular concentrations from the range from 0.001-10% by weight, based on the total weight of the formulation.

If vitamin A or vitamin A derivatives, or carotenes or derivatives thereof are the additional antioxidant or antioxidants, it is advantageous to choose their particular concentrations from the range from 0.001-10% by weight, based on the total weight of the formulation.

According to the invention, niacinimide and/or panthenol can also be used advantageously in the preparations.

Preparations according to the invention can also comprise active ingredients which stimulate circulation of the skin and/or have a vasodilatory effect, caffeine, B vitamins (pyridoxin and nicotinic acid and its derivatives), camphor, capsaicin being advantageous.

Moreover, selected formulations according to the invention which comprise, for example, antiwrinkle active ingredients, such as flavone glycosides (in particular α-glycosylrutin), coenzyme Q10, vitamin E and/or derivatives and the like are exceptionally advantageously suitable for the prophylaxis and treatment of cosmetic or dermatological changes in the skin, as arise, for example, during skin ageing (e.g. lines and wrinkles).

If α-glycosylrutin is the antioxidant, it is advantageous to choose its particular concentrations from the range from 0.001-5% by weight, based on the total weight of the formulation.

However, other pharmaceutically or dermatologically effective substances, such as, for example, substances which calm and care for the skin, can be incorporated into the preparations according to the invention. These include, for example, panthenol, allantoin, tannin, antihistimines, antiphlogistics, glucocorticoids (e.g. hydrocortisone), and plant active ingredients, such as azulene and bisabolol, glycyrrhizin, hamamelin, and plant extracts, such as camomile, aloe vera, hamamelis, liquorice.

Moisturizers is the term used to refer to substances or mixtures of substances which impart to cosmetic or dermatological preparations the property of, following application or distribution on the surface of the skin, reducing the moisture release of the horny layer (also called transepidermal water loss (TEWL)) and/or positively influencing hydration of the horny layer.

Advantageous moisturizers for the purposes of the present invention are, for example, glycerol, lactic acid and/or lactates, in particular sodium lactate, butylene glycol, propylene glycol, biosaccharide gum-1, glycine soya, ethylhexyloxyglycerol, pyrrolidonecarboxylic acid and urea. In addition, it is particularly advantageous to use polymeric moisturizers from the group of water-soluble and/or water-swellable and/or water-gellable polysaccharides. Of particular advantage are, for example, hyaluronic acid, chitosan and/or a fucose-rich polysaccharide, which is filed in the Chemical Abstracts under the registry number 178463-23-5 and is available, for example, under the name Fucogel®1000 from SOLABIA S.A. Moisturizers can advantageously also be used as antiwrinkle active ingredients for the prophylaxis and treatment of cosmetic or dermatological changes in the skin, as arise, for example, during skin ageing.

According to the invention, the following moisturizers are particularly preferably used: glycerol, sorbitol, urea and panthenol.

Here, it is advantageous according to the invention to use one or more moisturizers in a total concentration of from 1 to 25% by weight, based on the total weight of the preparation, in the emulsion.

The cosmetic or dermatological emulsions according to the invention can also advantageously, but not necessarily, comprise fillers which, for example, further improve the sensory and cosmetic properties of the formulations and, for example, bring about or enhance a velvety or silky feel on the skin. Advantageous fillers for the purposes of the present invention are starch and starch derivatives (such as, for example, tapioca starch, distarch phosphate, aluminium or sodium starch octenyl succinate and the like), pigments which have neither primarily UV filter nor colouring effect (such as, for example, boron nitride etc.) and/or AEROSILS® (CAS No. 7631-86-9).

According to the invention, the emulsions according to the invention can advantageously be used as ointment, cream or lotion. Their use in the form of a spray, e.g. a pump spray, is also advantageous according to the invention, in which case the preparations can advantageously also be foamed. Last but not least, according to the invention they can be used advantageously for impregnating a substrate (e.g. a nonwoven wipe).

The examples below are intended to illustrate the present invention without limiting it. Unless stated otherwise, all amounts, fractions and percentages are based on the weight and the total amount or on the total weight of the preparations.

### EXAMPLES

W/O emulsions

| | Example | | | | |
|---|---|---|---|---|---|
| | 1 | 2 | 3 | 4 | 5 |
| Triglycerol | 1.0 | 0.5 | 1.4 | 2.0 | 3.0 |
| diisostearate | | | | | |
| PEG-40 Perisostearate | 1.0 | 1.5 | 1.75 | 3.0 | 2.0 |
| Isopropyl palmitate | — | 3.0 | 3.0 | 5.0 | — |
| Paraffin oil | 12.5 | 10.0 | 8.5 | 5.0 | 1.5 |
| Vaseline | 8.0 | 6.0 | 3.0 | 12.0 | 2.5 |
| Silicone oil | 2.0 | 1.0 | 3.0 | 5.0 | 0.25 |
| (cyclomethicone) | | | | | |
| Isohexadecane | 0.5 | 0.75 | 3.0 | 2.0 | 0.25 |
| Octyldodecanol | 0.5 | 1.0 | 0.75 | 3.0 | 0.25 |
| Aluminium stearate | 0.4 | 0.3 | 0.6 | 1.0 | 0.05 |
| Dicaprylyl carbonate | 0.1 | 0.05 | 0.15 | 0.5 | 1.0 |
| Vitamin E acetate | 0.5 | 0.75 | 0.5 | 2.5 | 0.5 |
| Cocoa butter | 0.5 | 1.0 | 0.5 | 2.5 | 25 |
| Shea butter | 0.5 | 1.0 | 0.5 | 0.25 | 2.5 |
| Magnesium sulphate | 0.5 | 0.6 | 0.6 | 0.7 | 1.0 |
| Glycerol | 3.0 | 5.0 | 5.0 | 15.0 | 1.5 |
| Lactic acid | 0.2 | 0.1 | 0.7 | 0.3 | 1.0 |
| Citric acid | 0.1 | 0.25 | 0.05 | 0.1 | 0.2 |
| Sodium lactate | 0.2 | 0.05 | 1.5 | 0.3 | 2.0 |
| Perfume | q.s. | q.s. | q.s. | q.s. | q.s. |
| Caprylic/capric | 2.0 | 2.5 | — | 5.0 | — |
| triglyceride | | | | | |
| Potassium sorbate | 0.04 | 0.15 | 0.05 | 0.03 | 0.4 |
| Wool wax | 0.3 | 0.4 | 0.1 | 0.15 | — |
| Talc | — | — | 0.1 | 1.0 | 0.1 |
| Water | ad 100 | ad 100 | ad 100 | ad 100 | ad 100 |

W/O emulsions

| | Example | | | | |
|---|---|---|---|---|---|
| | 6 | 7 | 8 | 9 | 10 |
| PEG-30 | — | 0.5 | 0.25 | — | 3.0 |
| dipolyhydroxystearate | | | | | |
| Lanolin alcohol | 1.0 | 1.5 | 1.75 | 3.0 | — |
| Paraffin oil | 12.5 | 10.0 | 8.0 | 5.0 | 1.5 |
| Vaseline | 8.0 | 6.0 | 5.0 | 12.0 | 2.5 |
| Hydrogenated | 2.0 | 1.0 | 2.5 | 5.0 | 0.25 |
| cocoglycerides | | | | | |
| Hydrogenated | 0.5 | 0.75 | 1.0 | 2.0 | 0.25 |
| polyisobutene | | | | | |
| Octyldodecanol | 0.5 | 1.0 | 0.75 | 3.0 | 0.25 |
| Aluminium stearate | 0.4 | 0.3 | 0.6 | 1.0 | 0.05 |
| Dicaprylyl carbonate | 0.1 | 0.05 | 0.15 | 0.5 | 1.0 |
| Hydrogenated | 0.5 | 0.75 | 1.0 | 2.5 | 5.0 |
| castor oil | | | | | |
| Cocoa butter | 0.5 | 1.0 | 5.0 | 2.5 | 25 |
| Shea butter | 0.5 | 1.0 | 5.0 | 0.25 | 2.5 |
| Microcrystalline | 0.5 | 1.0 | 0.75 | 0.25 | 0.1 |
| cellulose | | | | | |
| Magnesium sulphate | 0.5 | 0.6 | 0.5 | 0.7 | 1.0 |
| Glycerol | 3.0 | 5.0 | 10.0 | 15.0 | 1.5 |
| Citric acid | 0.2 | 0.1 | 0.2 | 0.3 | 1.0 |
| Perfume | q.s. | q.s. | q.s. | q.s. | q.s. |
| 1,3-butylene glycol | 2.0 | — | 5.0 | — | — |
| Caprylic/capric | 2.0 | 2.5 | 3.0 | 5.0 | 0.5 |
| triglyceride | | | | | |
| Sodium dehydracet | — | — | 0.05 | — | — |
| Potassium sorbate | 0.3 | 0.4 | 0.25 | 0.15 | — |
| Talc | — | — | 0.05 | — | 0.1 |
| Water | ad 100 | ad 100 | ad 100 | ad 100 | ad 100 |

W/S emulsions

| | Example | | | | |
|---|---|---|---|---|---|
| | 11 | 12 | 13 | 14 | 15 |
| Cetyl PEG/PPG-10/1 | 1.0 | — | — | 3.0 | 5.0 |
| dimethicone | | | | | |
| Cylomethicone + | 10.0 | 12.5 | 25 | — | — |
| PEG/PPG-18/18 | | | | | |
| dimethicone (90:10) | | | | | |
| Cyclomethicone | 12.5 | 15 | 28.0 | 25.0 | 7.5 |
| Dimethicone | 5.0 | 13.0 | 5.0 | 12.0 | 5.0 |
| Hydrogenated | 0.5 | 0.75 | 1.0 | 2.0 | 0.25 |
| polyisobutene | | | | | |
| Octyldodecanol | 0.5 | 1.0 | 0.75 | 3.0 | 0.25 |
| Panthenol | 0.5 | 1.0 | 0.75 | 0.25 | 0.1 |
| Sodium chloride | 2.0 | 0.6 | 2.5 | 0.7 | 1.0 |
| Cocoa butter | 0.5 | 1.0 | 5.0 | 2.5 | 25 |
| Shea butter | 0.5 | 1.0 | 5.0 | 0.25 | 2.5 |
| Glycerol | 3.0 | 5.0 | 10.0 | 15.0 | 1.5 |
| Citric acid | 0.2 | 0.1 | 0.2 | 0.3 | 1.0 |
| Sodium citrate | 1.0 | 0.1 | 0.4 | 0.9 | 2.5 |
| Perfume | q.s. | q.s. | q.s. | q.s. | q.s. |
| Potassium sorbate | 0.4 | 0.1 | 0.05 | 0.3 | 0.4 |
| Microcrystalline | 1.0 | 0.1 | 0.5 | 0.25 | 0.1 |
| cellulose | | | | | |
| Cetyldimethicone | 0.5 | — | 0.7 | — | — |
| Benzyl alcohol | — | — | 0.05 | — | 0.1 |
| Modified starch | — | 2.5 | — | 0.15 | — |
| Water | ad 100 | ad 100 | ad 100 | ad 100 | ad 100 |

W/O emulsions

| | Example | | | | |
|---|---|---|---|---|---|
| | 16 | 17 | 18 | 19 | 20 |
| Cetyl PEG/PPG-10/1 | 1.0 | — | — | 3.0 | 5.0 |
| dimethicone | | | | | |
| Cylomethicone + | 10.0 | 12.5 | 25 | — | — |
| PEG/PPG-18/18 | | | | | |
| dimethicone (90:10) | | | | | |
| Cyclomethicone | 12.5 | 15 | 28.0 | 25.0 | 7.5 |
| Dimethicone | 5.0 | 13.0 | 5.0 | 12.0 | 5.0 |
| Hydrogenated | 0.5 | 0.75 | 1.0 | 2.0 | 0.25 |
| polyisobutene | | | | | |
| Octyldodecanol | 0.5 | 1.0 | 0.75 | 3.0 | 0.25 |
| Cocoa butter | 0.5 | 1.0 | 5.0 | 2.5 | 25 |
| Shea butter | 0.5 | 1.0 | 5.0 | 0.25 | 2.5 |
| Panthenol | 0.5 | 1.0 | 0.75 | 0.25 | 0.1 |
| Sodium chloride | 2.0 | 0.6 | 2.5 | 0.7 | 1.0 |
| Glycerol | 3.0 | 5.0 | 10.0 | 15.0 | 1.5 |
| Lactic acid | 0.2 | 0.1 | 0.2 | — | — |
| Sodium lactate | 0.2 | 1.0 | 0.05 | — | — |
| Perfume | q.s. | q.s. | q.s. | q.s. | q.s. |
| Microcrystalline | 1.0 | 0.1 | 1.5 | 2.5 | 0.1 |
| cellulose | | | | | |
| Potassium sorbate | 0.3 | 0.4 | 0.25 | 0.15 | — |
| Stearyldimethicone | 0.5 | — | 0.7 | — | — |
| Dehydracetic acid | — | — | 0.05 | — | 0.1 |
| Modified starch | — | 2.5 | — | 0.15 | — |
| Water | ad 100 | ad 100 | ad 100 | ad 100 | ad 100 |

W/O emulsions

| | Example | | | | |
|---|---|---|---|---|---|
| | 21 | 22 | 23 | 24 | 25 |
| PEG-22 Dodecyl | 5.0 | 1.5 | 0.25 | — | 3.0 |
| glycol copolymer | | | | | |
| PEG-45 Dodecyl | 1.0 | 1.5 | 1.75 | 3.0 | — |
| glycol polymer | | | | | |
| Paraffin oil | 12.5 | 10.0 | 8.0 | 5.0 | 7.5 |
| Isopropyl stearate | 8.0 | 6.0 | 5.0 | 12.0 | 2.5 |
| Hydrogenated | 2.0 | 1.0 | 2.5 | 5.0 | 0.25 |
| cocoglycerides | | | | | |
| Evening primrose | 0.5 | 0.75 | 1.0 | 2.0 | 0.25 |
| oil | | | | | |
| Octyldodecanol | 0.5 | 1.0 | 0.75 | 3.0 | 0.25 |
| Aluminium stearate | 0.4 | 0.3 | 0.6 | 1.0 | 0.05 |
| Cocoa butter | 0.5 | 1.0 | 5.0 | 2.5 | 25 |
| Shea butter | 0.5 | 1.0 | 5.0 | 0.25 | 2.5 |
| Dicaprylyl carbonate | 0.1 | 0.05 | 0.15 | 0.5 | 1.0 |
| Hydrogenated | 0.5 | 0.75 | 1.0 | 2.5 | 5.0 |
| castor oil | | | | | |
| Microcrystalline | 0.5 | 1.0 | 0.75 | 0.25 | 0.1 |
| cellulose | | | | | |
| Magnesium sulphate | 0.5 | 0.6 | 0.5 | 0.7 | 1.0 |
| Glycerol | 3.0 | 5.0 | 10.0 | 15.0 | 1.5 |
| Sodium citrate | 0.2 | 0.1 | — | — | — |
| Citric acid | 0.2 | 0.1 | — | — | — |
| Perfume | q.s. | q.s. | q.s. | q.s. | q.s. |
| 1,3-Butylene glycol | 2.0 | — | 5.0 | — | — |
| Caprylic/capric | 2.0 | 2.5 | 3.0 | 5.0 | 0.5 |
| triglyceride | | | | | |
| Potassium sorbate | 0.4 | 0.15 | 0.05 | 0.3 | 0.4 |
| Talc | 0.3 | 0.4 | 0.25 | 0.15 | — |
| Benzyl alcohol | — | — | 0.05 | — | 0.1 |
| Water | ad 100 | ad 100 | ad 100 | ad 100 | ad 100 |

W/O emulsions

| | Example | | | | |
|---|---|---|---|---|---|
| | 26 | 27 | 28 | 29 | 30 |
| Diglycerol | 3.0 | — | 0.25 | — | 3.0 |
| dipolyhydroxystearate | | | | | |
| Triglycerol | 1.0 | 3.5 | 1.75 | 2.5 | — |
| diisostearate | | | | | |
| PEG-40 sorbitan | — | 2.5 | 0.5 | 3.5 | 3.0 |
| isostearate | | | | | |
| Paraffin oil | 12.5 | 10.0 | 8.0 | 5.0 | 7.5 |
| Isopropyl stearate | 8.0 | 6.0 | 5.0 | 12.0 | 2.5 |
| Cocoa butter | 0.5 | 1.0 | 5.0 | 2.5 | 25 |
| Shea butter | 0.5 | 1.0 | 5.0 | 0.25 | 2.5 |
| Hydrogenated | 2.0 | 1.0 | 2.5 | 5.0 | 0.25 |
| cocoglycerides | | | | | |
| Isopropyl palmitate | 0.5 | 1.0 | 0.75 | 3.0 | 0.25 |
| Dicaprylyl carbonate | 0.1 | 0.05 | 0.15 | 0.5 | 1.0 |
| Hydrogenated | 0.5 | 0.75 | 1.0 | 2.5 | 5.0 |
| castor oil | | | | | |
| Microcrystalline | 0.5 | 1.0 | 0.75 | 0.25 | 0.1 |
| cellulose | | | | | |
| Magnesium sulphate | 0.5 | 0.6 | 0.5 | 0.7 | 1.0 |
| Glycerol | 3.0 | 5.0 | 10.0 | 15.0 | 1.5 |
| Citric acid | 0.2 | 0.1 | 0.1 | 0.3 | 1.0 |
| Sodium citrate | 0.2 | 0.3 | 0.2 | 1.5 | 0.8 |
| Perfume | q.s. | q.s. | q.s. | q.s. | q.s. |
| Caprylic/capric | 2.0 | 2.5 | 3.0 | 5.0 | 0.5 |
| triglyceride | | | | | |
| Potassium sorbate | 0.24 | 0.15 | 0.05 | 0.3 | 0.4 |
| Talc | — | — | 0.5 | — | 0.1 |
| Water | ad 100 | ad 100 | ad 100 | ad 100 | ad 100 |

That which is claimed:

1. A cosmetic or dermatological water-in-oil emulsion, comprising a lipid combination of shea butter and cocoa butter.

2. The emulsion according to claim 1, wherein the shea butter is present in an amount of from 0.1 to 30% by weight, based on the total weight of the emulsion.

3. The emulsion according to claim 1, wherein the cocoa butter is present in an amount of from 0.1 to 30% by weight, based on the total weight of the emulsion.

4. The emulsion according to claim 1, wherein the weight ratio of shea butter to cocoa butter in the emulsion is from 1:1 to 1:10.

5. The emulsion according to claim 1, further comprising one or more water-in-oil emulsifiers selected from the group consisting of polyglycerol isostearates and ethoxylated water-in-oil emulsifiers.

6. The emulsion according to claim 5, wherein the water-in-oil emulsifiers are present in a total amount of from 0.1 to 15% by weight, based on the total weight of the emulsion.

7. The emulsion according to claim 1, wherein the lipid combination is present in an amount effective for the prophylaxis, treatment or care of dry or cracked skin.

8. The emulsion according to claim 7, wherein the lipid combination is present in an amount effective for the prophylaxis, treatment or care of dry or cracked, highly pigmented skin.

9. The emulsion according to claim 1, wherein the lipid combination is present in an amount effective for the prophylaxis, treatment or care of ashy skin in people with highly pigmented skin.

10. A method for the prophylaxis, treatment or care of dry or cracked skin, or of ashy skin, comprising applying to the skin cosmetic or dermatological water-in-oil emulsion, comprising a lipid combination of shea butter and cocoa butter.

11. The method according to claim 10, for the prophylaxis, treatment or care of dry or cracked skin in people with highly pigmented skin.

12. The method according to claim 11, for the prophylaxis, treatment or care of dry or cracked skin in people having skin types of category IV-VI.

13. The method according to claim 10, for the prophylaxis, treatment or care of ashy skin in people with highly pigmented skin.

14. The method according to claim 13, for the prophylaxis, treatment or care of dry or cracked skin in people having skin types of category IV-VI.

15. The method according to claim 10, wherein the shea butter is present in an amount of from 0.1 to 30% by weight, based on the total weight of the emulsion.

16. The method according to claim 10, wherein the cocoa butter is present in an amount of from 0.1 to 30% by weight, based on the total weight of the emulsion.

17. The method according to claim 10, wherein the weight ratio of shea butter to cocoa butter in the emulsion is from 1:1 to 1:10.

18. The method according to claim 10, wherein the emulsion further comprises one or more water-in-oil emulsifiers selected from the group consisting of polyglycerol isostearates and ethoxylated water-in-oil emulsifiers.

19. The method according to claim 18, wherein the water-in-oil emulsifiers are present in a total amount of from 0.1 to 15% by weight, based on the total weight of the emulsion.
